(19) United States
(12) Patent Application Publication
(10) Pub. No.: US 2026/0165760 A1
(43) Pub. Date: Jun. 18, 2026

(54) TRIPHASIC BONE-CONNECTIVE TISSUE IMPLANT

(71) Applicant: University of Louisville Research Foundation, Inc., Louisville, KY (US)

(72) Inventors: Srimanta Barui, Louisville, KY (US); Kunal Kate, Louisville, KY (US)

(21) Appl. No.: 19/388,594

(22) Filed: Nov. 13, 2025

Related U.S. Application Data
(60) Provisional application No. 63/719,813, filed on Nov. 13, 2024.

(51) Int. Cl. A61B 17/86

(52) U.S. Cl. CPC A61B 17/8645; A61B 17/866

(57) ABSTRACT

A triphasic implant may include a bone phase zone defining a bone pore geometry. A triphasic implant may include a connective tissue phase zone defining a connective tissue pore geometry. A triphasic implant may include a transition phase zone including both the bone pore geometry and the connective tissue pore geometry.

### CROSS-REFERENCE TO RELATED APPLICATION

This application claims priority to and benefit of U.S. provisional patent application Ser. No. 63/719,813 filed Nov. 13, 2024, which is fully incorporated by reference.

### BACKGROUND

This disclosure relates to medical implants. More specifically, this disclosure relates to implants for bone-tendon or bone-ligament junctions.

### SUMMARY

In some aspects, the techniques described herein relate to a triphasic implant, including: a bone phase zone defining a bone pore geometry; a connective tissue phase zone defining a connective tissue pore geometry; and a transition phase zone including both the bone pore geometry and the connective tissue pore geometry.

In some aspects, the techniques described herein relate to a triphasic implant, wherein the triphasic implant is an interference screw.

In some aspects, the techniques described herein relate to a triphasic implant, wherein an overall length of the triphasic implant is 20 mm to 38 mm.

In some aspects, the techniques described herein relate to a triphasic implant, wherein an overall diameter of the triphasic implant is 6 mm to 14 mm.

In some aspects, the techniques described herein relate to a triphasic implant, wherein the bone pore geometry defines a pore size of 800 μm to 1200 μm.

In some aspects, the techniques described herein relate to a triphasic implant, wherein the bone pore geometry defines a cuboidal or rectangular shape.

In some aspects, the techniques described herein relate to a triphasic implant, wherein the connective tissue pore geometry defines a pore width of 150 μm to 400 μm, and a pore length of 1000 μm to 1600 μm.

In some aspects, the techniques described herein relate to a triphasic implant, wherein the connective tissue pore geometry defines an acicular pore shape.

In some aspects, the techniques described herein relate to a triphasic implant, wherein the bone pore geometry and the connective tissue pore geometry are interconnected through the bone phase zone, the connective tissue phase zone, and the transition phase zone.

In some aspects, the techniques described herein relate to a triphasic implant, further including threads having a pitch of 1 mm to 4 mm.

In some aspects, the techniques described herein relate to a triphasic implant, further including threads having a thread height of 0.5 mm to 1.5 mm.

In some aspects, the techniques described herein relate to a triphasic implant, further including at least one of active bone-cutting threads, unidirectional buttress threads, and hybrid threads.

In some aspects, the techniques described herein relate to a triphasic implant, wherein a length of the transition phase zone is 10% to 20% of an overall length of the triphasic implant.

In some aspects, the techniques described herein relate to a triphasic implant, further including a screw head defining a screw head diameter equal to or less than a maximum diameter of the bone phase zone, the connective tissue phase zone, and the transition phase zone.

In some aspects, the techniques described herein relate to a triphasic implant, further including a guide wire channel defining a diameter of 0.75 mm to 1.5 mm.

In some aspects, the techniques described herein relate to a triphasic implant, further including an inner drive cavity defining a homogeneous cross-section throughout an inner length.

In some aspects, the techniques described herein relate to a triphasic implant, further including an inner drive cavity defining a tapered cross-section that is narrower toward the connective tissue phase zone.

In some aspects, the techniques described herein relate to a triphasic implant, further including an inner drive cavity defining a depth that is 50% to 75% of an overall length of the triphasic implant.

In some aspects, the techniques described herein relate to a triphasic implant, further including an inner drive cavity defining cross-sectional shape that is triangular, tetragonal, pentagonal, hexagonal, heptagonal, or octagonal.

In some aspects, the techniques described herein relate to a triphasic implant, wherein the triphasic implant is constructed of a biomaterial including titanium alloys, biomedical grade steels, cobalt-chromium, magnesium, or strontium doped bioceramics including stabilized zirconia, hydroxyapatite/TCP doped zirconia, zirconia toughened alumina, PEEK, PLA, PLLA, hydroxyapatite/TCP doped PLA, PLLA, PDLLA, or PLGA.

In some aspects, the techniques described herein relate to a triphasic implant, wherein the triphasic implant is manufactured using a conventional machining method, or CAD-CAM, or advanced additive manufacturing.

In some aspects, the techniques described herein relate to a triphasic implant, wherein the triphasic implant is modified using metallic ions.

In some aspects, the techniques described herein relate to a triphasic implant, wherein the bone phase zone is modified with Cu2+ ion.

In some aspects, the techniques described herein relate to a triphasic implant, wherein the connective tissue phase zone is modified using Zn2+ ion.

In some aspects, the techniques described herein relate to a triphasic implant, wherein the transition phase zone is modified with both Cu2+ ion and Zn2+ ion.

In some aspects, the techniques described herein relate to a triphasic implant, wherein ion modification can be carried out in a dual-chamber reactor with flowing ionic solutions.

In some aspects, the techniques described herein relate to a triphasic implant, wherein ion modification can be carried out by sequential dip-coating.

In some aspects, the techniques described herein relate to a triphasic implant, wherein ion modification includes a copper sulfate solution and a zinc sulfate solution.

This summary is illustrative only and is not intended to be in any way limiting. Other aspects, features, and advantages of the devices or processes described herein will become apparent in the detailed description set forth herein, taken in conjunction with the accompanying figures, wherein like reference numerals refer to like elements.

### BRIEF DESCRIPTION OF DRAWINGS

The device is explained in even greater detail in the following drawings. The drawings are merely exemplary and certain features may be used singularly or in combination with other features. The drawings are not necessarily drawn to scale.

FIGS. 1A and 1B are schematic diagrams showing an innovative tissue implant approach, according to some implementations. The schematic diagram shows 3D printing of YSZ-based triphasic tissue implants to mimic the natural bone-tendon structure, and post-processing methods, including heat treatment and the introduction of bimetallic ions (Cu2+ and Zn2+), to enhance zone-specific tissue regeneration.

FIG. 2 is a series of perspective views of biomimetic bone-tendon interfacial structures derived triphasic interference screws with different clinically practiced diameters (not limited to the numbers shown in the figure), according to some implementations.

FIG. 3 is a detail view of a triphasic interference screw with the bone, tendon/ligament and the transition areas clearly distinguishable, according to some implementations. The bone phase is characterized by isotropic cuboidal porosities whereas acicular and fibrous porosity contributes the ligament/tendon part of the screw. There are 10-15% overlap in the two zones making the interface. The implants have hybrid threads of one directional buttress threads in the proximal zone (bone) and aggressive V-threads in the distal side (ligament/tendon). While the aggressive threads facilitate the bone cutting and insertion, buttress threads resist the axial pull out under tensile force. The implants also consist of K-Wire channel and long tapered hex driver groove. The groove cross section can be triangular, square, pentagonal and hexagonal.

FIGS. 4A and 4B each illustrate a series of photographs of a prototype of a ceramic interference screw (alpha-prototype) manufactured with SLA-based manufacturing, before and after high temperature sintering, according to some implementations.

FIG. 5 is a schematic representation of a dual-chamber reactor system, according to some implementations.

FIG. 6 illustrates an interference screw CAD, according to some implementations, of an illustrative proof-of-principle example.

FIGS. 7A and 7B illustrate examples of interference screws printed and sintered by Lithoz where FIG. 7A shows a full screw, and FIG. 7B shows a half screw.

### DETAILED DESCRIPTION

Following below are more detailed descriptions of concepts related to, and implementations of, methods, apparatuses, and systems for a triphasic bioceramic bone-tendon/ligament implant. The figures illustrate exemplary implementations in detail and the present disclosure is not limited to the details or methodology set forth in the description or illustrated in the figures. The terminology used herein is for the purpose of description only and should not be regarded as limiting.

As used herein, connective tissue may refer to tendons, ligaments, or other fibrous connective tissues that facilitate movement and stability in a patient's body. While specific reference may be made to a tendon, a ligament, a bone-tendon/ligament interface, junction, or other relationship, it should be noted that other connective tissue types are contemplated within the scope of this disclosure. In other words, reference to a tendon is not exclusive of applicability to ligaments, and vice-versa. All connective tissues are contemplated herein, even when specific reference is made during explanation of exemplary implementations.

Tendon-bone procedures in musculoskeletal healthcare are common and often require revisions. For instance, there was a 22% increase in ACL reconstruction rates between 2002 and 2014, with 20-25% of ACL reconstruction patients needing revisions within 7-10 years. The large rate of required revisions highlights a need for an improved implant at a bone-tendon interface. Current bone-tendon implants are typically made from materials like titanium and Polyether-ether-ketone (PEEK) using injection molding, but they face challenges. Metallic implants can release heavy metal ions, potentially leading to immune reactions and complications, and are prone to stress shielding due to mechanical property mismatches with host-bone at the bone-tendon interface. Meanwhile, polymeric implants often lack the required tissue-specific microstructures, and bioresorbable polymer implants hinder monitoring through traditional X-ray techniques, causing post-surgery complications. Moreover, the macroscopic tri-phasic nature of the bone-tendon structure, unaddressed by current implants, requires a deeper understanding of how bone-tendon porosity variation among different patient demographics influences mechanical behavior. Such insights are crucial for defining implant properties and design.

The dynamic microstructures of the bone-tendon interface and/or a bone-ligament interface and the mechanical property gradients extending from bone to tendon/ligament present formidable hurdles with far-reaching implications for individuals and society. The long-term repair of torn or dislocated bone-tendon/bone-ligament junctions through conventional re-insertion and suturing has shown limited success. Conventional ‘monolithic’ polymeric and metallic screws also pose challenges, including stress shielding, bioinertness-related adverse immune responses, radio-transparency issues, and metallosis-related clinical complications. As described below, a bioceramic-based matrix system in the form of a triphasic interference screw is capable of biomimetically restoring the functional bone-tendon/bone-ligament tissue complex. Bioceramics are popular for their mechanical strength and biocompatibility, osteo-inductivity, host-integration capabilities, and the facilitation of enhanced bone remodeling in vivo.

The bio-inspired ‘triphasic’ bone-tendon tissue interface described herein addresses the existing issues in the technology and addresses long standing needs by using cutting-edge 3D printing technologies, encompassing bone, bone-tendinous junction, and tendon analogs, in compliance with near-net-shape fabrication standards. One aspect of implementing a triphasic implant involves the strategic deployment of high-resolution additive manufacturing techniques, coupled with post-processing treatments that involve zone-specific modification of tissue-engineered architectures using bimetallic ions via a specially designed reactor. This approach enhances the functionality of osteoblasts and tenocytes, promoting the regeneration of a biomimetic tissue complex facilitating a seamless transition between the two dissimilar tissue types in the interfacial region. The impact of the development disclosed herein extend into the realm of public health by reducing the cost burden associated with the treatment of musculoskeletal disorders and injuries, for example in ACL reconstruction in sports medicine. This is achieved through tissue-specific biomimetic design and surface modifications to facilitate superior host-integration and improved bio-functionalities, which in turn reduce the likelihood of implant failure and the need for revision surgeries.

Production of the triphasic implant includes combining additive manufacturing (e.g., 3D printing technology) with bioceramics, offering a potential solution to the limitations of conventional metallic and polymeric implants. However, the full potential of bioceramics like Yttria-Stabilized Zirconia (YSZ), known for its excellent osteoinductive, osteoconductive, and host-integration capabilities, remains untapped due to a lack of understanding and difficulties in ceramic 3D printing with tailorable designs and mechanical properties. The triphasic implants disclosed herein employ 3D printing to craft YSZ-based biomimetic tri-phasic structures for bone-tendon restorative implants. This disclosure addresses the existing knowledge gaps by investigating how these structures perform under both mechanical and biological conditions. The inventors have tested prototypes of topological and biochemical features identified in laboratory-scale triphasic bone-tendon scaffolds and prototyped triphasic interference screws using the technology described herein. This advancement is poised to revolutionize the field of musculoskeletal healthcare, offering enhanced and personalized solutions that will make a significant contribution to the public health sector.

Referring to the figures generally, the various implementations disclosed herein relate to systems, apparatuses, and methods for a triphasic interference implant that includes a bone phase zone defining a bone pore geometry, a connective tissue phase zone defining a connective tissue pore geometry, and a transition phase zone defining a blend or mixture of bone pore geometry and connective tissue pore geometry. The triphasic interference implant can be provided as a screw implant. In some implementations, the screw implant includes cutting threads that accelerate insertion, and one-directional buttress threads that inhibit pull-out of the implant. In some implementations, the triphasic interference implant is produced via additive manufacturing (e.g., 3D printing) a matrix, and treating the matrix in a reactor. In some implementations, the reactor provides zone specific biochemical modifications of the matrix. For example, a first treatment is applied to the bone phase zone, a second treatment is applied to the connective tissue phase zone, and a blend of the first treatment and the second treatment is applied to the transition phase zone.

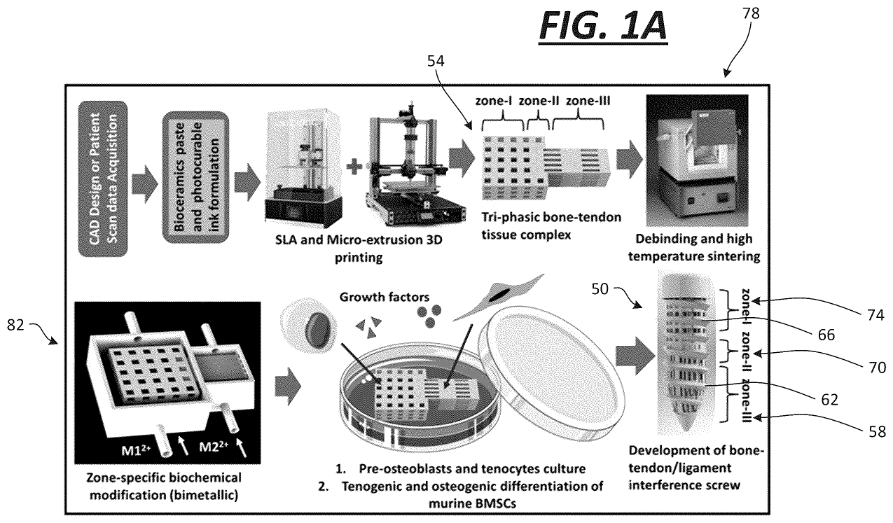

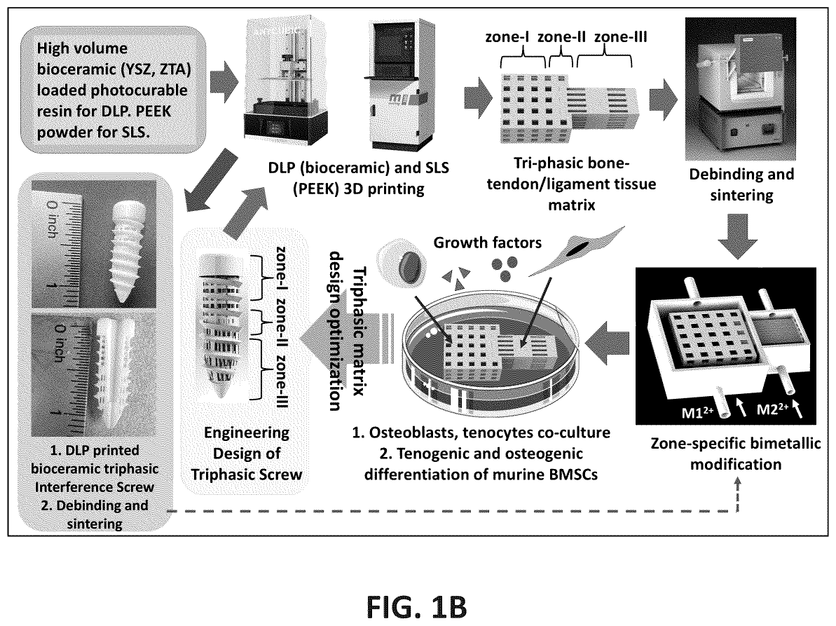

As shown in FIG. 1A, a triphasic implant in the form of a triphasic interference screw 50 includes three conceptual categories (i.e., verticals) in bio-inspired interference screw development. Firstly, the development of a triphasic tissue complex 54 with biomimetic bone-tendon structure manufactured by utilizing 3D printing with yttria stabilized zirconia (YSZ) with a specific focus on replicating the topological and biomechanical properties of natural bone, tendon, and a bone-tendon junction. Secondly, based on the triphasic bone-tendon interfacial structure, the triphasic interference screw 50 is designed with clinically relevant length and diameter. In addition, the triphasic interference screw 50 includes a self-tapping apical end 58 having aggressive bone-cutting threads 62 as well as unidirectional buttress threads 66 (e.g., having the same pitch and height) on a middle portion 70 and proximal end 74. Thirdly, post-processing of the triphasic tissue complex 54 after 3D printing is chosen including heat treatment strengthening 78 and bone and tendon zone-specific bimetallic ion (e.g., Cu2+ and Zn2+) modification using a dual-chamber reactor system 82, to enhance cell-material interactions and tissue regeneration. This approach enables advanced and most-critical tissue-complex regeneration. Furthermore, successful implementations of the triphasic interference screw 50 promote the use of bioceramics (e.g., YSZ) based bone-tendon implants in view of the well-established superior osteoinductive and host-integration capabilities of bioceramics compared to the traditional metallic and plastic implants. The triphasic interference screw 50 showcases a unique convergence of advanced manufacturing techniques and biomaterial science and engineering to revolutionize this segment of musculoskeletal regenerative medicine.

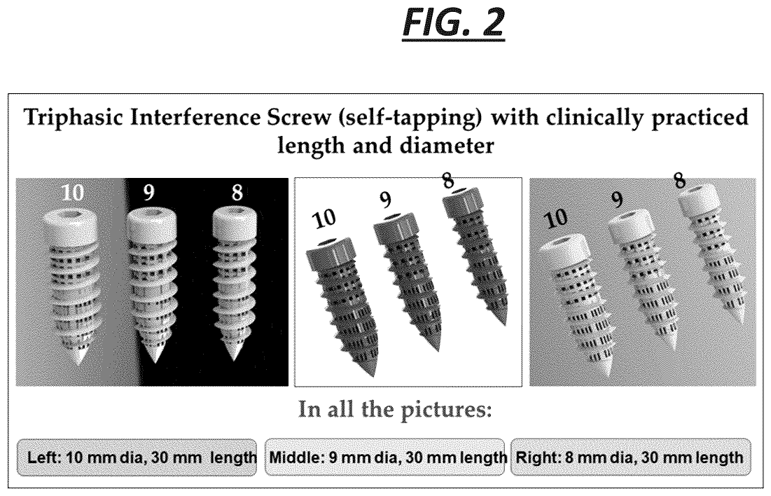

To create the prototype triphasic interference screw 50, three primary factors were considered. First, optimal osteoblastic activities occur in the pore size range of 500 to 1500 μm. Second, tenocyte (typical tendon fibroblasts) activities thrive in acicular porosities with a high aspect ratio (1:5→1:10). Third, a 10-15% overlap between the tenocyte phase and the osteoblastic phase simulates the bone-tendon interface. It is important to provide 100% interconnected porosities at the bone and tendon tissue interface. This interconnectivity provides a continuous pathway for tenocytes and osteoblasts to integrate with the implant. In FIG. 1B, one of the 3D printing route is changed from micro-extrusion to SLS (selective laser sintering) of PEEK polymer. The major manufacturing route is unchanged to DLP/SLA of bioceramics. The real photographs are from DLP 3D printed bioceramic (zirconia) triphasic implants. SLA is stereolithography route very often used interchangeably with DLP in production, although there is a technical difference on how the laser scans the layer. Employing this same microstructural gradient, different sized and geometries of triphasic interference screws 50 (e.g., clinically practiced diameters, as depicted in FIG. 2, homogeneous threads) can be provided. Additionally, other shaped implants can be provided utilizing the triphasic tissue complex 54. The clinically acceptable lengths and diameters also vary in a broader range which is not shown in FIG. 2 (i.e., other shapes and sizes are contemplated).

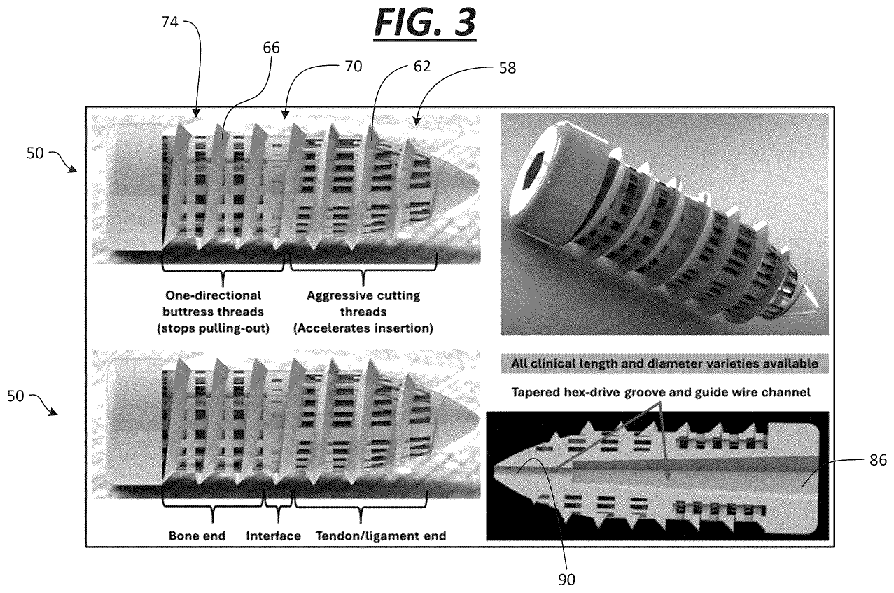

As shown in FIG. 3, the hybrid nature of the threads includes the aggressive bone-cutting threads 62 in the distal/apical region 58 (i.e., the ligament/tendon end) and the unidirectional buttress threads 66 having identical thread pitch and height in the proximal/bone region 74. The aggressive threads 62 accompanied with the self-tapping end 58 facilitates a seamless insertion experience in a pilot drilled hole in an implantation site. The buttress threads 66 inhibit any potential implant pull-out under tensile force exerted by a re-inserted tendon/ligament under physiological activities. The triphasic interference screw 50 also includes a deep tapered hex drive socket 86 that enables effective insertion torque and driving experience while a guide wire channel in the form of a through K-guide wire channel 90 leads the implantation direction.

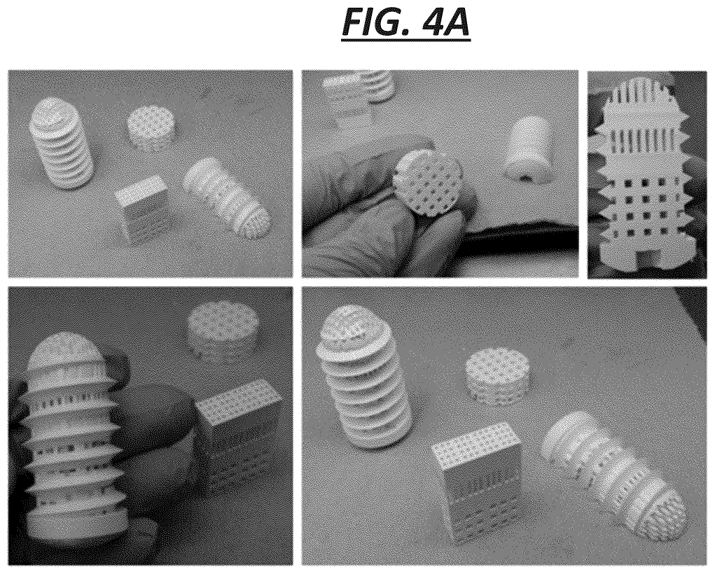

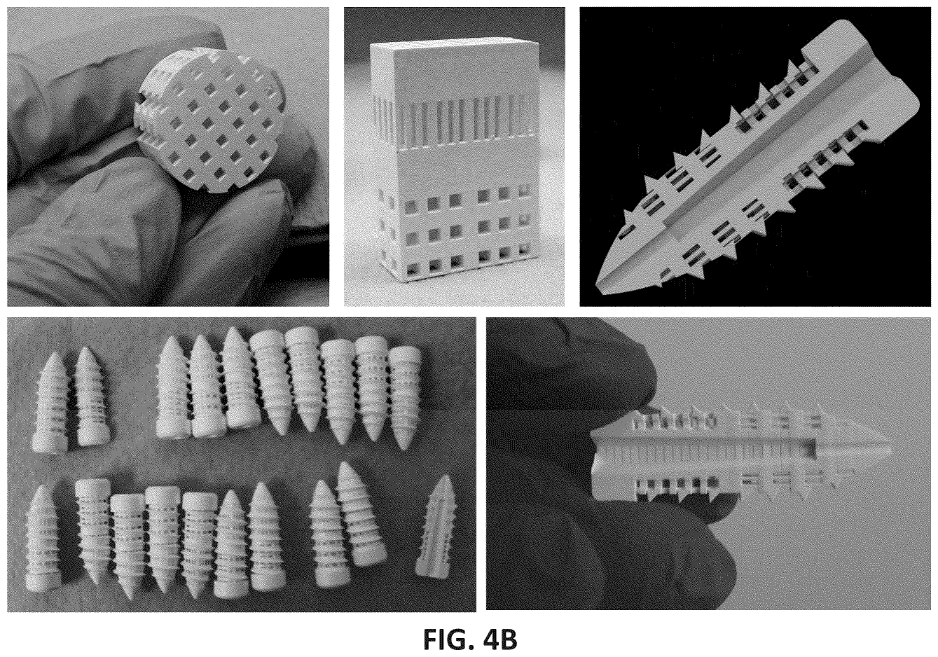

As shown in FIG. 4A, prototype triphasic tissue complexes and prototype screws were manufactured and subjected to mechanical property assessments (e.g., compressive, tensile, flexural) and cell culture experiments using multiple cells lines (e.g., osteoblasts, tenocytes, BMSCs stem cells). The prototypes included complexes similar to the triphasic tissue complexes 54 discussed above. FIG. 4B illustrates prototype triphasic tissue complexes and prototype screws

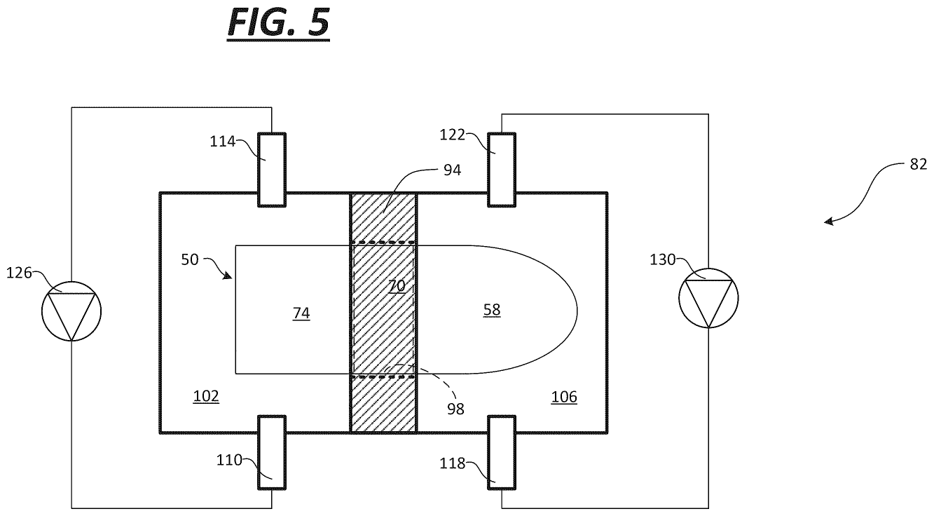

As shown in FIG. 5, to modify the surface of the triphasic interference screw 50 in a zone-specific manner, we developed the dual-chamber reactor system 82 (e.g., a silicone based dual chamber reactor). The dual-chamber reactor system 82 includes a separating barrier 94 having the thickness of the intermediate zone 70 of the triphasic interference screw 50. The separating barrier 94 defines a reactor opening 98 in the separating barrier 94, tight enough to only pass the triphasic interference screw 50 through it. After placement, the proximal end 74 (i.e., a bone end) will remain open in a first chamber 102 and the self-tapping apical end 58 (i.e., a tendon end) in a second chamber 106, while the middle portion 70 tightly passes through reactor opening 98 of the separating barriers 94. The first chamber 102 includes a first inlet 110 and a first outlet 114. The second chamber 106 includes a second inlet 118 and a second outlet 122. A first pump 126 (e.g., a peristaltic pump) flows an aqueous copper sulphate solution through the first chamber 102. A second pump 130 (e.g., a peristaltic pump) flows an aqueous zinc sulphate solution through the second chamber 106. While the bone and tendon sides of the porous scaffolds are getting infiltrated by the Cu2+ and Zn2+ ions, slight diffusion of these ion through the interconnected designed and process (binder evaporation, sintering) induced microporosities lead to mixing in the middle portion 70 (i.e., a transitional zone) of the triphasic interference screw 50. After the slow infiltration for 4-6 hours in the dual-chamber reactor system 82, the triphasic interference screw 50 scaffold is taken out of the dual-chamber reactor system 82 and lyophilized. The resulting triphasic interference screw 50 includes three distinct chemical environments comprising Cu2+ in the proximal end 74 (i.e., bone end), Zn2+ self-tapping apical end 58 (i.e. tendon end), and the middle portion 70 (i.e., intermediate mixed zone) includes both the ions representing the transitional phase from bone to tendon.

Based on the interference screws invented and displayed in FIG. 3, the broad workable ranges of several characteristic parameters of the implant screws are provided herein. In some implementations, the overall length of the interference screws can vary in the range of 20 mm to 38 mm. In some implementations, the overall diameter of the interference screws can vary in the range of 6 mm to 14 mm. In some implementations, the bone side pore size can range from 800 μm to 1200 μm (cuboidal to rectangular). In some implementations, the tendon/ligament side pore breadth and width ranges from 150 μm to 400 μm, pore length can vary around 1000 μm to 1600 μm (acicular pore shape). In some implementations, the pores in the interference screw are hybrid type (for bone and tendon), or single type (either cuboidal, for bone or acicular, for tendon). The interference screws can be solid also with no pores in the structure. In some implementations, all the pores are partially or fully interconnected. In some implementations, the interference screw thread pitch can vary from 1 to 4 mm. In some implementations, the thread height can vary form 0.5 mm to 1.5 mm. In some implementations, the length of the intermediate bone to ligament/tendon overlapping zone can vary in the range of 10% to 20% with respect to the overall length of the screw. In some implementations, the screw head portion should be of the same diameter of the screw implant. The screw can be without any head portion also, having the threads throughout the length of the screw. In some implementations, the guide wire channel diameter can vary from 0.75 mm to 1.5 mm. In some implementations, the inner drive cavity can be of homogeneous cross-section throughout the inner length, or tapered (distal narrow and proximal wider). In some implementations, the depth of the tapered drive cavity can range from 50% to 75% of the overall length of the screw. In some implementations, the shape of the inner cavity for tapered drive can vary from triangular, tetragonal, pentagonal, hexagonal, heptagonal and octagonal. In some implementations, the thread types can vary from all active (bone-cutting) or all ‘unidirectional buttress’ or hybrid in nature having both types of threads. In some implementations, the biomaterial used to fabricate the interference screws can vary from titanium alloys, biomedical grade steels, cobalt-chromium, magnesium, strontium doped bioceramics like stabilized zirconia, hydroxyapatite/TCP doped zirconia, zirconia toughened alumina, PEEK, PLA, PLLA, hydroxyapatite/TCP doped PLA, PLLA, PDLLA, or PLGA. In some implementations, the interference screws can be manufactured using conventional machining method or CAD-CAM (for non-porous) or advanced additive manufacturing (for homogeneous or hybrid porous implants). In some implementations, the screws can be modified using metallic ions to enhance the host tissue integration. In a non-limiting example, the bone side can be modified with Cu2+ ion whereas the ligament/tendon end can be modified using Zn2+ ion. The intermediate transition zone can comprise both the ions. In some implementations, the ion modification can be carried out in dedicated dual-chamber reactor with flowing ionic solutions or simply sequential dip-coating in the corresponding ionic solutions. In a non-limiting example, the ionic solution can be copper sulfate and zinc sulfate to modify the bone and tendon zones respectively.

### Non-Limiting Example

Anterior Cruciate Ligament (ACL) reconstruction relies on interference screws, but existing designs often require revision surgeries. This project presents a novel interference screw design, 3D printed using zirconia ceramics. We utilized Lithoz for zirconia ceramic 3D printing of interference screws and used it as a control to develop an in-house binder system for printing with a liquid resin-based Digital Light Processing (DLP) printer. Our findings highlighted the impact of printing process on the screw fabrication. The screws printed using Lithoz were sintered at 1400° C.-1450° C. Same sintering protocol was followed for all the parts, including screws, printed using in-house DLP process. We analyzed density, sintering characteristics, and the mechanical properties of the parts printed by our method, demonstrating a viable pathway for in-house DLP printing of zirconia for interference screws. This research advances ceramic 3D printing for biomedical applications, offering a promising alternative for ACL reconstruction implants.

### 1. INTRODUCTION

Anterior cruciate ligament (ACL) reconstruction is among the most common orthopedic sports medicine procedures performed annually in the United States, with estimates ranging from 100,000 to 200,000 surgeries per year [1][2]. The incidence of ACL reconstructions has increased significantly over time, with data showing a 22% rise from 61.4 per 100,000 person-years in 2002 to 74.6 per 100,000 person-years in 2014 [3 ]. This upward trend is particularly pronounced among adolescents aged 13-17 years, who have experienced large increases in both isolated ACL reconstructions (37%) and those with concomitant meniscal repairs (107%) [3].

Despite advances in surgical techniques and implant technology, the failure rate following ACL reconstruction remains substantial. Studies indicate that 10-25% of ACL reconstruction patients require revision surgeries within 7-10 years of their primary procedure [4, 5]. More concerning is that following revision surgery, failure rates up to 30% have been reported in the literature [6], with approximately 49% of revision patients ultimately needing a second revision procedure. These statistics highlight the critical need for improved implant technologies to enhance positive surgical outcomes and reduce the burden of multiple surgeries and the associated complications.

The standard clinical procedure for ACL reconstruction involves creating bone tunnels in the femur and tibia, pulling a ligament or tendon graft through these tunnels, and securing the graft under appropriate tension. For graft fixation, interference screws have emerged as the most clinically popular devices since they were first introduced by Lambert in 1983[7]. These screws function by compressing the graft against the wall of the bone tunnel, providing immediate fixation strength until biological integration occurs. Currently, two major categories of interference screws dominate the market: bioresorbable and nonresorbable variants. Non-resorbable screws are typically fabricated from metallic materials (titanium alloy, stainless steel) or bioinert polymers such as poly-ether-ether-ketone (PEEK). Bioresorbable alternatives are predominantly manufactured using polymers such as poly-L-lactic acid (PLLA) or poly-lactic acid-coglycolic acid (PLGA), sometimes combined with osteoconductive materials like β-tricalcium phosphate (β-TCP) or hydroxyapatite (HAp) [8].

However, each of these material options presents significant limitations. Metallic screws can cause metallosis, stress shielding, graft laceration during insertion, and complications during magnetic resonance imaging (MRI) [1]. Bioresorbable polymer screws often exhibit uncontrolled degradation rates, insufficient mechanical properties, and are prone to breakage during insertion [8]. Magnesium-based screws, while offering promising osteoconductivity, can lead to gas cavity formation in surrounding tissues as they degrade, potentially delaying bone healing [9].

A critical barrier to the success of ACL reconstruction is the limited understanding of the role interference screws play in bone-to-ligament/tendon interfacial healing rather than merely serving as temporary anchoring devices. Additionally, the commercial market and scientific literature lacks biomimetically structured interference screws designed specifically to enhance this biological integration process [1].

To address these limitations, we propose exploring ceramic-based interference screws, specifically those manufactured from zirconia. Zirconia-based implants offer several advantages compared to existing solutions, including mechanical properties that more closely mimic natural bone, excellent corrosion resistance, and superior osteoconductivity without the drawbacks associated with metallic or polymeric alternatives. Zirconia exhibits transformation toughening behavior, which improves its fracture resistance under mechanical loading. Additionally, its bioinert nature minimizes inflammatory responses, making it a favorable candidate for orthopedic applications. This paper presents our development of 3D-printed zirconia interference screws using both commercial (Lithoz) and in-house digital light processing (DLP) printing systems. We evaluate the manufacturability of complex screw geometries and assess the viability of these approaches for creating functional interference screws that may improve outcomes in ACL reconstruction procedures.

### 2. METHODOLOGY

### 2.1 Materials

For this study, we utilized yttria-stabilized zirconia (YSZ) powder for 3D printing of interference screws. The zirconia powder was sourced from [Stanford Advanced Materials], with specific particle size distribution and purity level suitable for DLP printing.

### 2.2 Interference Screw Design

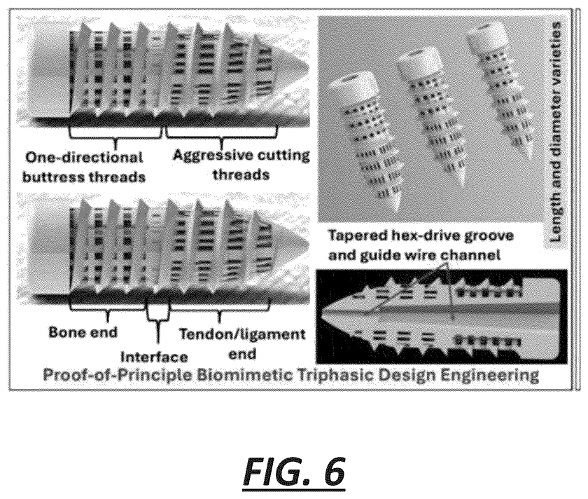

The interference screws were designed using SolidWorks CAD software with dimensions based on commercially available screws, including diameters ranging from 6-10 mm and lengths of 20-30 mm. The screws were designed with hybrid thread features, including aggressive cutting threads at the distal end and unidirectional buttress threads at the proximal/bone region to prevent pull-out. A tapered hex-drive socket was incorporated to enable engagement with an insertion tool, and a through channel was included for K-guide wire, as shown in FIG. 6.

### 2.3 Ceramic Slurry Preparation

For the in-house DLP printing, photocurable ceramic slurries were prepared by mixing zirconia powder with a photopolymerizable resin system. The resin system comprised: a resin with primary monomer; a crosslinking agent; a photo-initiator; and a ceramic dispersant to prevent agglomeration.

The zirconia powder was gradually added to the resin mixture while stirring to achieve homogeneous dispersion. The solid loading was optimized to achieve suitable viscosity for DLP printing while maximizing the ceramic content. For comparison, commercial Lithoz CeraFab slurries were also used according to the manufacturer's specifications.

### 2.4 3D Printing Process

### 2.4.1 Commercial Lithoz Printing

Interference screws were printed using a Lithoz CeraFab printer following the manufacturer's recommended parameters with layer thicknesses of approximately 25-50 μm.

### 2.4.2 In-House DLP Printing

For the in-house method, we used an Asiga DLP printer equipped with 405 nm UV light sources. The printing parameters were optimized with specific attention to: Layer height (25 μm); and Exposure time per layer (30 seconds).

### 2.5 Post-Processing

### 2.5.1 Debinding

The printed green parts underwent a thermal debinding process to remove the organic components. The debinding cycle was determined based on thermogravimetric analysis (TGA) of the ceramic-resin system.

The typical debinding protocol comprised: Slow heating (0.5-1° C./min) to 200° C.; Hold for 1 hour; Continued heating to 400° C.; Hold for 2 hours; Final ramp to 600° C.; and Total debinding time: 12-15 hours.

### 2.5.2 Sintering

After debinding, the parts were sintered in a high-temperature furnace. For zirconia, sintering occurred at 1400-1450° C. for 2-3 hours. Heating and cooling rates were carefully controlled (3-5° C./min) to prevent thermal shock and cracking. The sintering atmosphere in the furnace was air for all of the samples.

### 2.6 Characterization

### 2.6.1 Density Measurement

The density of sintered parts was measured using the Archimedes method. The relative density was calculated as a percentage of the theoretical density of zirconia.

### 2.6.2 Dimensional Analysis

The dimensional accuracy was evaluated by measuring key dimensions using digital calipers and comparing them to the original CAD design. Shrinkage factors were calculated to assess the predictability of the manufacturing process.

### 2.6.3 Mechanical Testing

Mechanical properties of the sintered screws were evaluated through insertion torque measurement.

### 3. RESULTS

### 3.1 Printability and Process Optimization

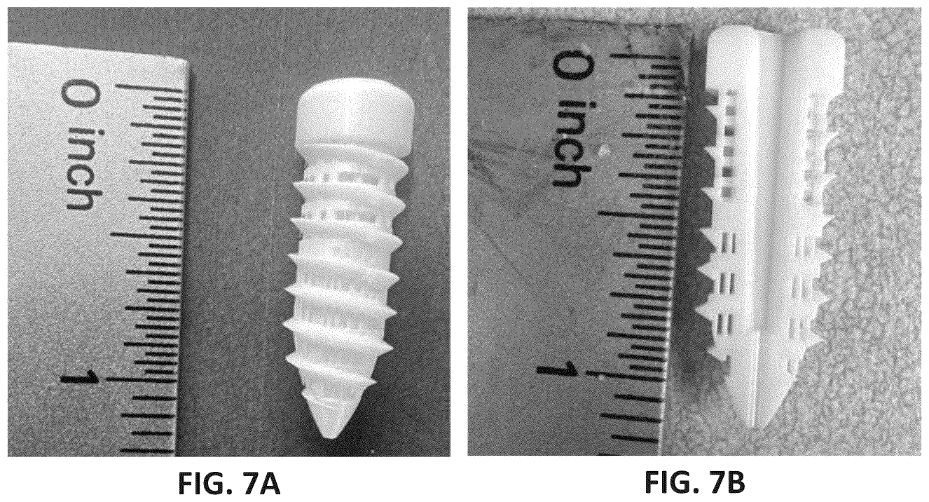

FIGS. 7A and 7B illustrate examples of interference screws printed and sintered by Lithoz where FIG. 7A shows a full screw, and FIG. 7B shows a half screw. These screws exhibited excellent dimensional stability which can be observed in the half screw. These screws were kept as standard to achieve accurate printing in DLP printers.

Cubes were printed first to achieve the best path to debinding and sintering. Then, porous scaffolds were designed, printed, debinded, and sintered as a precursor to the complex interference screws. After that interference screws were printed using the DLP printer.

Our DLP-printed zirconia screws demonstrated the ability to reproduce complex geometries, including fine thread features and internal channels. Challenges encountered included: 1. Maintaining homogeneity of the ceramic slurry during printing; 2. Preventing light scattering effects that reduced resolution in dense suspensions; 3. Controlling layer adhesion to ensure structural integrity; 4. Managing layer delamination and large crack formation during debinding; and 5. Managing shrinkage during sintering to maintain dimensional accuracy.

### 3.2 Physical Properties of Sintered Parts

The screws printed and sintered by Lithoz achieved around 99% of Zirconia's density (5.68 g/cc). On the other hand, the parts printed by DLP printer achieved 94% of that density. So, density-wise the DLP printed parts need more improvements. Zirconia cubes with an average edge length of 8.0 mm were printed in DLP printer. Following thermal debinding, the parts exhibited a dimensional reduction to an average of 7.15 mm, resulting in a shrinkage of approximately 10.63%.

A subsequent sintering process further reduced the average dimension to 5.7 mm, representing an additional shrinkage of approximately 20.28% relative to the debinded state. Overall, the total shrinkage from the green to the sintered state was calculated to be 28.75%, indicating an effective densification process happening during sintering.

Moreover, the screws printed by Lithoz have better surface finish and dimensional stability compared to the screws printed by DLP printer. More slurry modifications and printing parameter modifications might be needed to achieve better surface finish and dimensional stability.

### 3.3 Mechanical Properties

The mechanical properties of the sintered zirconia screws include: Insertion torque values; and Pull-out force. For this article, only the insertion torque test has been done using torque gauge (Torbal FS Torque). The screws were put into 15 PCF polyurethane foam.

The torque values are within the range required for clinical application in ACL reconstruction, with the modulus being closer to that of natural bone compared to metallic alternatives. The highest torque recorded was 1.5 N·m. This is well below the limit of 3 N·m for orthopedic applications [10]. The average value was recorded within 0.5-1 N·m.

### 3.4 Comparison Between Commercial and In-House Printing

The commercial Lithoz system generally produced parts with higher density and better surface finish, but our in-house DLP system demonstrated comparable mechanical properties and adequate feature resolution at a lower cost per part. More works on different printing parameters as well as the debinding process are expected to give higher densities with better surface finish and thus reducing the difference between the Lithoz prints and the DLP prints.

### 4. CONCLUSIONS

This study demonstrates the successful 3D printing of zirconia interference screws using both commercial and in-house DLP systems. The achievements include: 1. Development of a printable zirconia slurry suitable for in-house DLP printing. 2. Successful reproduction of complex screw geometries with functional threads and complicated porous features. 3. Achievement of mechanical properties suitable for ACL reconstruction applications. 4. Demonstration of a cost-effective alternative to commercial ceramic printing systems.

The zirconia interference screws offer a promising alternative to traditional metallic and polymeric options, with potential advantages in biocompatibility, mechanical properties, and integration with host tissue. The in-house DLP printing approach provides a pathway for broader adoption and customization of ceramic medical devices for orthopedic applications.

### 6. REFERENCES

Unless otherwise indicated, the following publications are fully incorporated by reference and made a part hereof to show the state of the art at the time of the present invention:
[1] A. D. Booher, Design and Evaluation of a Novel Porous Interference Screw for
Ligament Reconstruction, M.S. thesis, Dept. of Biomedical Engineering, Wake Forest Univ., Winston-Salem, NC, USA, 2019.
[2] N. A. Mall, P. J. Chalmers, G. J. Moric, B. D. Tanaka, C. D. Cole, and B. J. Bach, “Incidence and trends of anterior cruciate ligament reconstruction in the United States,” The American Journal of Sports Medicine, vol. 42, no. 10, pp. 2363-2370, 2014.
[3] M. M. Herzog, S. W. Marshall, J. L. Lund, V. Pate, C. D. Mack, and J. T. Spang, “Trends in incidence of ACL reconstruction and concomitant procedures among commercially insured individuals in the United States, 2002-2014,” Sports Health, vol. 10, no. 6, pp. 523-531, 2018.
[4] R. W. Magnussen, K. A. Lawrence, K. A. West, and K. P. Toth, “Long-term failure of anterior cruciate ligament reconstruction,” Arthroscopy: The Journal of Arthroscopic & Related Surgery, vol. 29, no. 10, pp 1566-1571, October 2013.
[5] B. Wang and K. T. Lee, “Results of revision anterior cruciate ligament reconstruction using a transportal technique,” Acta Orthopaedica Belgica, vol. 81, no. 4, pp. 752-758, 2015.
[6] B. Wang and K. T. Lee, “Results of revision anterior cruciate ligament reconstruction using a transportal technique,” Acta Orthopaedica Belgica, vol. 81, no. 4, pp. 752-758, 2015.
[7] K. A. Feldman, “The principles of interference screw fixation: Application to foot and ankle surgery,” Techniques in Foot & Ankle Surgery, vol. 4, no. 1, pp. 52-60, March 2005.
[8] S. Adnan, “Improving biodegradable interference screw properties by combining polymers,” Clinical Publications, November 2022. [Online].
[9] N. O. Ikegami, H. Takechi, H. Yamamoto, T. Fujisawa, and H. Matsugaki, “Development of a model system for gas cavity formation behavior of biodegradable magnesium alloy screws,” Materials, vol. 15, no. 11, pp. 3854, June 2022.
[10] P. Siroros, L. M. Stippler, A. B. A. Silva, D. E. Haas, and A. B. Imhoff, “In vitro investigation of the fixation performance of a bioabsorbable magnesium ACL interference screw compared to a conventional interference screw,” Life, vol. 13, no. 2, p. 484, February 2023.

### Conclusion

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

Features disclosed in this specification (including any accompanying claims, abstract, and drawings), and/or all of the steps of any method or process so disclosed, may be combined in any combination, except combinations where at least some of such features and/or steps are mutually exclusive. The claimed features extend to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

The terms “coupled”, “connected”, and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

Certain terminology is used in the following description for convenience only and is not limiting. The words “right”, “left”, “lower”, and “upper” designate direction in the drawings to which reference is made. The words “inner” and “outer” refer to directions toward and away from, respectively, the geometric center of the described feature or device. The words “distal” and “proximal” refer to directions taken in context of the item described and, with regard to the instruments herein described, are typically based on the perspective of the practitioner using such instrument, with “proximal” indicating a position closer to the practitioner and “distal” indicating a position further from the practitioner. The terminology includes the above-listed words, derivatives thereof, and words of similar import.

Throughout the description and claims of this specification, the word “comprise” and variations of the word, such as “comprising” and “comprises”, means “including but not limited to”, and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the form disclosed. Many modifications and variations will be apparent to those of ordinary skill in the art without departing from the scope and spirit of the present disclosure.

What is claimed is:

1. A triphasic implant, comprising:
a bone phase zone defining a bone pore geometry;
a connective tissue phase zone defining a connective tissue pore geometry; and
a transition phase zone including both the bone pore geometry and the connective tissue pore geometry.

2. The triphasic implant of claim 1, wherein the triphasic implant is an interference screw.

3. The triphasic implant of claim 1, wherein an overall length of the triphasic implant is 20 mm to 38 mm.

4. The triphasic implant of claim 1, wherein an overall diameter of the triphasic implant is 6 mm to 14 mm.

5. The triphasic implant of claim 1, wherein the bone pore geometry defines a pore size of 800 μm to 1200 μm.

6. The triphasic implant of claim 1, wherein the bone pore geometry defines a cuboidal or rectangular shape.

7. The triphasic implant of claim 1, wherein the connective tissue pore geometry defines a pore width of 150 μm to 400 μm, and a pore length of 1000 μm to 1600 μm.

8. The triphasic implant of claim 1, wherein the connective tissue pore geometry defines an acicular pore shape.

9. The triphasic implant of claim 1, wherein the bone pore geometry and the connective tissue pore geometry are interconnected through the bone phase zone, the connective tissue phase zone, and the transition phase zone.

10. The triphasic implant of claim 1, further comprising threads having a pitch of 1 mm to 4 mm.

11. The triphasic implant of claim 1, further comprising threads having a thread height of 0.5 mm to 1.5 mm.

12. The triphasic implant of claim 1, further comprising at least one of active bone-cutting threads, unidirectional buttress threads, and hybrid threads.

13. The triphasic implant of claim 1, wherein a length of the transition phase zone is 10% to 20% of an overall length of the triphasic implant.

14. The triphasic implant of claim 1, further comprising a screw head defining a screw head diameter equal to or less than a maximum diameter of the bone phase zone, the connective tissue phase zone, and the transition phase zone.

15. The triphasic implant of claim 1, further comprising a guide wire channel defining a diameter of 0.75 mm to 1.5 mm.

16. The triphasic implant of claim 1, further comprising an inner drive cavity defining a homogeneous cross-section throughout an inner length.

17. The triphasic implant of claim 1, further comprising an inner drive cavity defining a tapered cross-section that is narrower toward the connective tissue phase zone.

18. The triphasic implant of claim 1, further comprising an inner drive cavity defining a depth that is 50% to 75% of an overall length of the triphasic implant.

19. The triphasic implant of claim 1, further comprising an inner drive cavity defining cross-sectional shape that is triangular, tetragonal, pentagonal, hexagonal, heptagonal, or octagonal.

20. The triphasic implant of claim 1, wherein the triphasic implant is constructed of a biomaterial including titanium alloys, biomedical grade steels, cobalt-chromium, magnesium, or strontium doped bioceramics including stabilized zirconia, hydroxyapatite/TCP doped zirconia, zirconia toughened alumina, PEEK, PLA, PLLA, hydroxyapatite/TCP doped PLA, PLLA, PDLLA, or PLGA.

21. The triphasic implant of claim 1, wherein the triphasic implant is manufactured using a conventional machining method, or CAD-CAM, or advanced additive manufacturing.

22. The triphasic implant of claim 1, wherein the triphasic implant is modified using metallic ions.

23. The triphasic implant of claim 22, wherein the bone phase zone is modified with Cu2+ ion.

24. The triphasic implant of claim 22, wherein the connective tissue phase zone is modified using Zn2+ ion.

25. The triphasic implant of claim 22, wherein the transition phase zone is modified with both Cu2+ ion and Zn2+ ion.

26. The triphasic implant of claim 22, wherein ion modification can be carried out in a dual-chamber reactor with flowing ionic solutions.

27. The triphasic implant of claim 22, wherein ion modification can be carried out by sequential dip-coating.

28. The triphasic implant of claim 22, wherein ion modification includes a copper sulfate solution and a zinc sulfate solution.
